(19) United States
(12) Patent Application Publication
(10) Pub. No.: US 2006/0166869 A1
(43) Pub. Date: Jul. 27, 2006

(54) Novel use of lipopeptide preparations

(76) Inventors: Magali Deleu, Sombreffe (BE); Robert Brasseur, Haillot (BE); Michel Paquot, Noville-sur-Mehaigne (BE); Herve Legros, Gembloux (BE); Samuel Dufour, Gembloux (BE); Philippe Jacques, Liege (BE); Jacqueline Destain, Chastre (BE); Philippe Thonart, Saint Denis (BE); Alain Brans, Beaufays (BE); Bernard Joris, Spa (BE); Jean-Marie Frerc, Nandrin (BE)

Correspondence Address: KNOBBE MARTENS OLSON & BEAR LLP, 2040 MAIN STREET, FOURTEENTH FLOOR, IRVINE, CA, 92614, US

(21) Appl. No.: 10/519,734

(22) PCT Filed: Jun. 27, 2003
(86) PCT No.: PCT/EP03/06817

§ 371 Date: Jul. 25, 2005

(30) Foreign Application Priority Data
Jun. 28, 2002 (EP) 02077567.2
Dec. 20, 2002 (EP) 02080663.4

(51) Int. Cl. A61K 38/12

(52) U.S. Cl. 514/9

(57) ABSTRACT

The invention relates to the use of lipopeptide preparations as anti-tilted-peptide agents, which are able to inhibit or limit the destabilization effect of tilted peptides on hydrophobic/hydrophilic interfaces, for instance on membranes. The lipopeptides include surfactins, iturins, and fengycins. The invention also relates to a process for the production of the lipopeptide preparations.

The present invention relates to a novel use of lipopeptide preparations.

Lipopeptides are molecules that have been known for years and have a lot of known functions, the primary of which is biosurfactants. It has been shown in document WO 00/29426 that some lipopeptides may have insecticidal, antifungal and antibacterial properties. Document DE 19633684 refers to a scientific article, from ITOKAWA H. et al, Chem. Pharm. Bull. 42, 604-607 (1994) that states that two surfactins, namely [IIe₇] and [Leu₇] surfactins show a moderate anti HIV-activity. This article effectively mentions such effect, but no tangible results are given and there is no scientific discussion on this activity. U.S. Pat. No. 5,801,143 show that some specific lipopeptides, namely cyclic depsipeptides may inhibit the production of apolipoprotein B.

As used herein, the following terms must be understood according to the definitions given below.

The term “membrane” refers to the hydrophili/hydrophobic interfaces which surround biological cells.

The term “tilted peptide” refers to peptide comprising 10 to 30 amino acids and presenting a helicoidal secondary structure with an axis forming an angle of 45±20° with respect to the plane of the membrane.

The term “anti-tilted-peptide agent” refers to molecules able to inhibit or limit the destabilisation effect of a tilted peptide on a hydrophobic/hydrophilic interface, for instance on membranes as mentioned above.

The term “lipopeptide” refers to a molecule having a cyclic or linear peptidic part and a lipidic part consisting in a fatty acid chain.

The term “lipopeptide preparation” refers to a preparation containing at least one lipopeptide, either alone or in combination with at least one other component.

The term “derivatives of lipopeptides” encompasses molecules in which at least a moiety has been modified, as for instance, esters of lipopeptide, amides of lipopeptide, sulfonated aminomethane derived of lipopeptides, lipopeptides with a different succession of amino acids, and the like.

The term “lipopeptide family” refers to a family of lipopeptide having all a common peptidic backbone and different lipidic parts having different carbon chain lengths and isomeries.

The term “crude lipopeptide mixture” refers to a preparation containing a mixture of surfactins, iturins and fengycins, each containing various homologous molecules having different fatty acid chain lengths and isomeries, as well as other molecules such as carbohydrates, amino acids, pigments, trace elements, the proportion of the other molecules being inferior to 25%.

The term “lipopeptide mixture” refers to a preparation containing lipopeptides of different families.

The term “lipopeptide homologous” refers to a lipopeptide of a given lipopeptide family having a specific number of carbon atoms and isomery in its fatty acid chain.

The term “aerobic conditions” relates to conditions, in a process for the production of lipopeptide preparation wherein the aeration rate is usual in the field

The term “microaerobic conditions” relates to conditions, in a process for the production of lipopeptide preparations, wherein the aeration rate is reduced vis-à-vis the aerobic conditions.

Disruption or destabilisation of a hydrophobic/hydrophilic interface is a common feature of several biological phenomena like virus fusion, lipid metabolism, neurotoxic mechanisms. One of the motifs involved in the mechanism is a tilted peptide. This peptide has the particularity to have a hydrophobicity gradient which gives it a tilted orientation in the lipid bilayer of a membrane, and is therefore referred to under the name of “tilted peptide”.

This kind of peptide has been found in proteins i.a. involved in the fusion of virus (like for example Simian and Human Immunodeficiency Virus, Ebola, Newcastle Disease virus, Bovine and Murine Leukaemia Virus, Influenza Virus) with the host cell, in lipid metabolism (lipolytica enzymes, apolipoproteins, . . . ), in signal sequences, in membrane proteins, in the fusion of spermatozoon with ovum and also in neurotoxic proteins involved in neurodegenerative diseases (like Alzheimer's disease).

The general characteristics of several tilted peptides from the literature, namely LINS et al, in PROTEINS: Structure, Function and Genetics 44: 435-447, 2001 and BRASSEUR, in Molecular Membrane Biology, 17, 31-40, 2000 are presented in Table 1.

In this table, under “Protein or Virus”, one will find the name of the protein or the virus in which the tilted peptide is detected. The following abbreviations are used:
SIV and HIV: respectively, Simian and Human Immunodeficiency Virus;
NDV: Newcastle Disease Virus;
1bct: bacteriorhodopsine;
BLV and MLV: respectively, Bovine and Murine Leukaemia Virus;
LCAT: Lecithin Cholesterol Acyl Transferase;
CETP: Cholesteryl Ester Transport Protein;
HLP: Hepatic Lipase;

LPL: Lipoprotein.

TABLE 1
General characteristics of several tilted peptides from the literature

| | Amino | Mapping | | |
|---|---|---|---|---|
| | acids | in the | | Protein |
| Protein or virus | number | sequence | Sequence | function |
| SIV | 12 | 528-539 | GVFVLGFLGFLA | Virus fusion |
| HIV | 12 | 478-489 | AVGIGALFLGFL | Virus fusion |
| β Amyloid | 14 | 29-42 | GAIIGLMVGGVVIA | Neurotoxic |
| β Amyloid | 12 | 29-40 | GAIIGLMVGGVV | Neurotoxic |
| Measles virus | 12 | | FAGVVLAGAALG | Virus fusion |
| NDV | 18 | 104-121 | FIGAIIGSVALGVATAAG | Virus fusion |
| Rous sarcoma virus | 17 | | FLGFLLGVGSAIASGVA | Virus fusion |
| 1bct | 16 | 177-192 | VTVVLWSAYPVVWLIG | Transmembr |
| 1bct | 18 | 195-212 | GAGIVPLNIETLLFMVLD | Transmembr. |
| Sendai virus | 17 | | FFGAVIGTIALGVATSA | Virus fusion |
| BLV | 12 | 269-280 | SPVAALTLGLAL | Virus fusion |
| MLV | 17 | | GPVSLTLALLLGGLTMG | Virus fusion |
| Yeast invertase | 19 | 1-19 | MLLQAFLFLLAGFAAKISA | peptide signal |
| Murine leukaemia virus | 17 | | GPVSLTLALLLGGLTMG | virus fusion |
| Ebola | 17 | 524-540 | GAAIGLAWIPYFGPAAE | virus fusion |
| Human prion | 18 | 118-135 | AGAVVGGLGGYMLGSAMS | Neurotoxic |
| LCAT | 13 | 56-68 | DFFTIWLDLNMFL | Transport |
| Influenza HA-2 | 20 | 1-20 | GLFGAIAGFIENGWEGMIDG | Virus fusion |
| ApoB 100 human precursor | 12 | | RPALLALLALPA | Signal peptide |
| Hepatite B, S protein | 16 | 1-16 | MENITSGFLGPLLVLQ | Virus fusion |
| Human Apo A-II Sakacin P | 13 | 58-70 | TELVNFLSYFVEL | Transport |
| CETP | 16 | 461-476 | FGFPEHLLVDFLQSLS | Transport |
| Meltrine | 14 | 591-603 | VIGTNAVSIETNIE | Myoblast fusion |
| Human Apo A-II | 18 | 53-70 | IKKAGTELVNFLSYFVEL | Transport |
| HLP | 13 | 234-246 | FLELYRHIAQHGF | lipase |
| LPL | 13 | 218-230 | IGEAIRVIAERGL | lipase |
| Fertiline | 17 | 83-99 | DSTKCGKLICTGISSIP | spermatzoid |

The international code for the representation of amino acids is used herein, either in the form of the one-letter code as used above or in the form of the three-letter code where appropriate. For the avoidance of doubt, the code as used is reproduced herein below in table 2.

TABLE 2
Amino acids code

| One-letter code | Amino acid name | Three-letters code |
|---|---|---|
| G | Glycine | Gly |
| P | Proline | Pro |
| A | Alanine | Ala |
| V | Valine | Val |
| L | Leucine | Leu |
| I | Isoleucine | Ile |
| M | Methionine | Met |
| C | Cysteine | Cys |
| F | Phenylalanine | Phe |
| Y | Tyrosine | Tyr |
| W | Tryptophan | Trp |
| H | Histidine | His |
| K | Lysine | Lys |
| R | Arginine | Arg |
| Q | Glutamine | Gln |
| N | Asparagine | Asn |
| E | Glutamic Acid | Glu |
| D | Aspartic Acid | Asp |
| S | Serine | Ser |
| T | Threonine | Thr |

Up to day, the diseases in which tilted peptides are involved remain extremely difficult to cure.

It has now been surprisingly found, according to the invention, that lipopeptide preparations may be used successfully as anti-tilted-peptide agents.

The lipopeptide preparations comprise lipopeptides which are selected from the group consisting of cyclic and linear lipopeptides, their homologous and derivatives and mixtures thereof.

The cyclic lipopeptides are preferably selected from the group consisting of surfactins, iturins and fengycins.

In a first embodiment of the invention, cyclic surfactin preferably have formula (I) 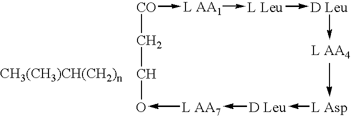
wherein the total number of carbon atoms in the fatty acid chain is comprised between 12 to 17, n being comprised between 6 and 11, AA₁ is Glu or Gln, AA₄ is Val or Ala and AA₇ is Val, IIe or Leu.

Preferably, n is comprised between 7 and 9, AA₁ is Glu, AA₄ is Val and AA₇ is Leu.

According to a particularly preferred embodiment of the invention, the cyclic surfactins are selected from the group consisting of an iso-branched β-hydroxyled fatty acid chain containing 13 carbon atoms (SC13), a surfactin with a linear β-hydroxyled fatty acid chain containing 14 carbon atoms (SC14), and a surfactin with an iso-branched β-hydroxyled fatty acid chain containing 15 carbon atoms (SC15).

According to a second embodiment of the invention, cyclic iturins have formula (II) 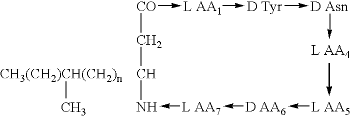
wherein the total number of carbon atoms in the fatty acid chain is comprised between from 13 to 17, n being comprised between 6 and 10, AA₁ is Asn or Asp, AA₄ is Gln, Pro or Ser, and AA₅ is Pro, Glu, or Gln, AA₆ is Asn or Ser, and AA₇ is Ser, Asn or Thr.

Preferably, the iturins are selected from the group consisting in an iturin wherein n is comprised between 7 and 10, AA₁ is Asn, AA₄ is Gln, AA₅ is Pro, AA₆ is Asn and AA₇ is Ser.

More preferably, the iturins are selected from the group consisting of a linear β-amino fatty acid chain containing 14 carbon atoms (IC14), an iturin with an iso-branched 62 -amino fatty acid chain containing 15 carbon atoms (IC15), an iturin with an iso-branched or linear β-amino fatty acid chain containing 16 carbon atoms (IC16), an iturin with an anteiso-branched β-amino fatty acid chain containing 17 carbon atoms (IC17)

According to a third embodiment of the invention, cyclic fengycins have formula (III) 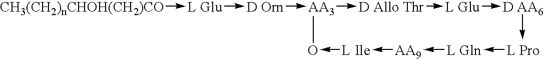
wherein the total number of carbon atoms in the fatty acid chain is comprised between from 12 to 18, n being comprised between 8 and 14, AA₃ is D Tyr or L Tyr, AA₆ is Val or Ala, and AA₉ is L Tyr or D Tyr.

Preferably, fengycin is fengycin A with a β-hydroxyled fatty acid chain containing 16 carbon atoms (FAC16), wherein AA₃ is D Tyr, AA₆ is Ala and AA₉ is L Tyr.

The invention also covers the use of lipopeptide preparations as anti-tilted peptide agents, wherein the linear lipopeptides are selected from the group consisting of surfactins, iturins and fengycins.

Each of the linear lipopeptides is obtainable by chemical modification of the corresponding cyclic lipopeptide.

In a particular embodiment, wherein the linear lipopeptides (LSC12 to LSC17) are linear surfactin that have formula (IV) 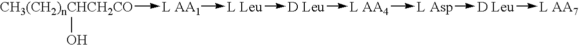
wherein the total number of carbon atoms in the fatty acid chain is comprised between 12 and 17, n being comprised between 8 and 13.

According to another particular embodiment, each of the linear lipopeptides is obtainable by chemical synthesis. In such a case, each of the linear lipopeptides (LSSC4 to LSSC24) has the formula (V) 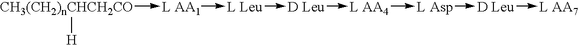
wherein the total number of carbon atoms in the fatty acid chain is 4 to 24; n being comprised between 0 and 20.

Preferably, AA₁ is Glu or Gln, AA₄ is Val or Ala and AA₇ is Val, IIe or Leu.; more preferably, AA₁ is Glu, AA₄ is Val and AA₇ is Leu.

It is preferred that the lipopeptide preparations comprise at least two lipopeptides, which may belong to different lipopeptide families.

In a particularly preferred lipopeptide preparations, one of the lipopeptides is selected from the group consisting of SC13 and SC15 and the other lipopeptide is FAC16.

The lipopeptides may have been obtained by a method chosen from biosynthesis by a micro-organism, chemical synthesis and chemical modifications of biosynthetised lipopeptides.

When they are obtained by a micro-organism, the latter may be chosen from the group consisting in Pseudomonas spp, Bacillus spp., Arthrobacter spp, Streptomyces spp., Serratia spp., Gluconobacter spp., and Agrobacterium spp, the species being for instance chosen from the group consisting of Bacillus subtilis, Bacillus licheniformis, and Bacillus globigii, Streptomyces aurantiacus, Arthrobacter MIS 38, Serratia marcescens, Gluconobacter cerinu, and Agrobacterium tumefaciens

Preferably, Bacillus subtilis is a strain chosen from the group consisting of ATCC 7067 and S499.

The invention also relates to a process for the production of a lipopeptide preparation which comprises an aerobic step followed by a microaerobic step. Preferably, this process produces a foam containing a concentrated mixture of different lipopeptide families

### DETAILED DESCRIPTION OF THE INVENTION

The invention will be further illustrated below by the description of some ways of carrying it out, with reference to the appended figures, wherein:

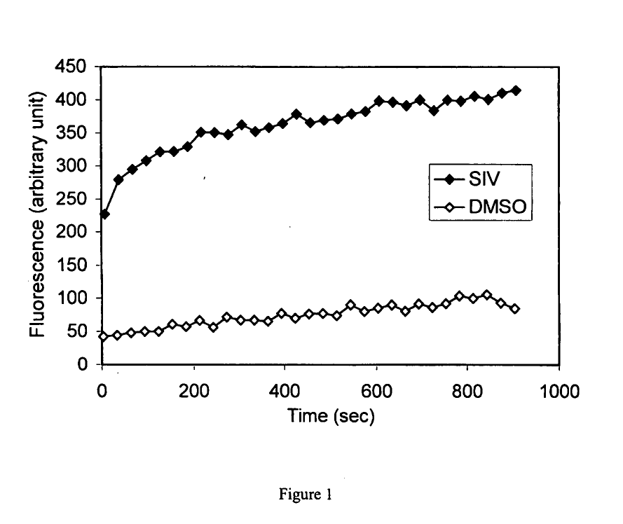

FIG. 1 represents the fluorescence intensity evolution as a function of time for large unilamellar vesicles (LUV) mixture in the presence of SIV tilted peptide at a concentration of 0.966 μM or in the presence of DMSO;

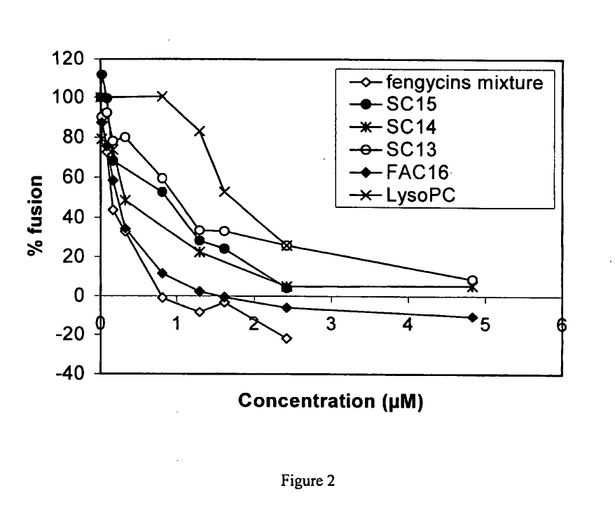

FIG. 2 represents the percentage of charged LUV fusion induced by SIV tilted peptide at a concentration of 0.966 μM in the presence of different concentrations of lipopeptides or lysophosphatidylcholine in a Tris NaCl buffer at pH 8.0.

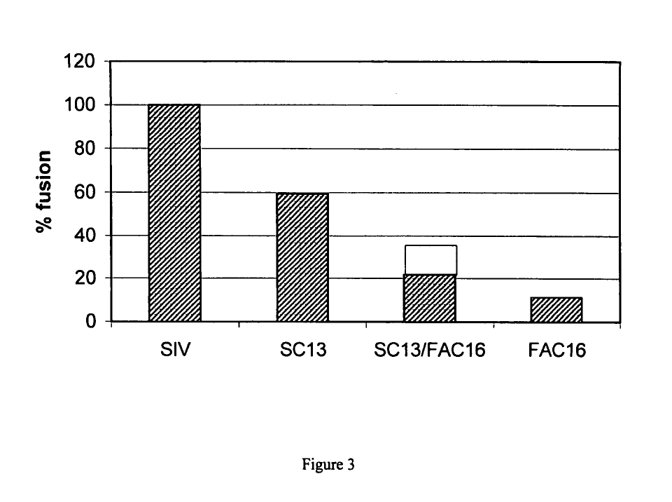

FIG. 3 represents the percentage of charged LUV fusion induced by SIV tilted peptide at a concentration of 0.966 μM in the presence of a mix of SC13 and FAC16 in proportion 1:1 at a concentration of 8.05×10⁻¹ μM in comparison with the activity of SC13 and FAC16 alone at the same concentration in a Tris NaCl buffer at pH 8.0.; the white rectangle represents the expected value.

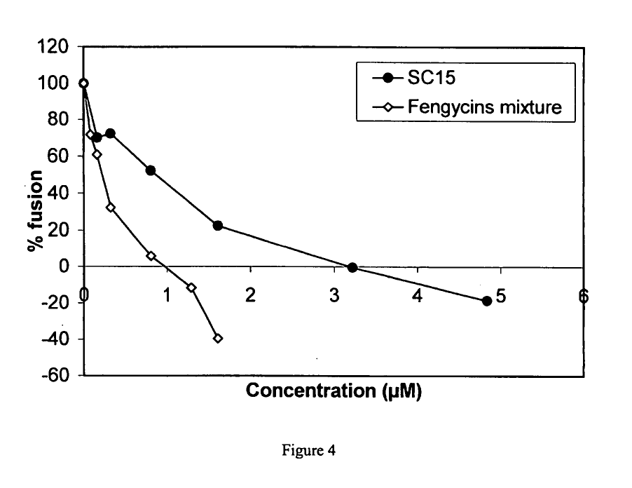

FIG. 4 represents the percentage of uncharged LUV fusion induced by SIV tilted peptide at a concentration of 0.966 μM in the presence of different concentrations of SC15 and fengycins mixture in a Tris NaCl buffer at pH 8.0.

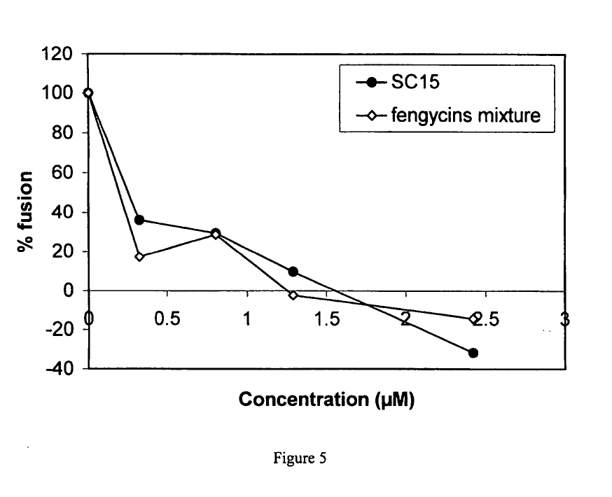

FIG. 5 represents the percentage of uncharged LUV fusion induced by SIV tilted peptide at a concentration of 0.966 μM in the presence of different concentrations of SC15 and fengycins mixture in a Tris buffer without NaCl at pH 8.0.

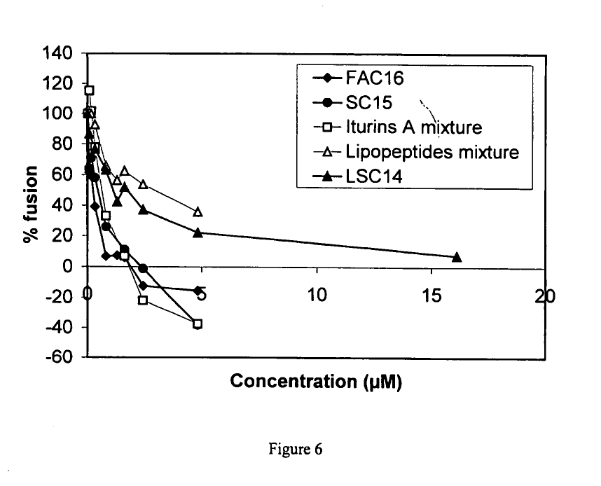

FIG. 6 represents the percentage of charged LUV fusion induced by SIV tilted peptide at a concentration of 0.966 μM in the presence of different concentrations of lipopeptides in a Tris NaCl buffer at pH 7.4.

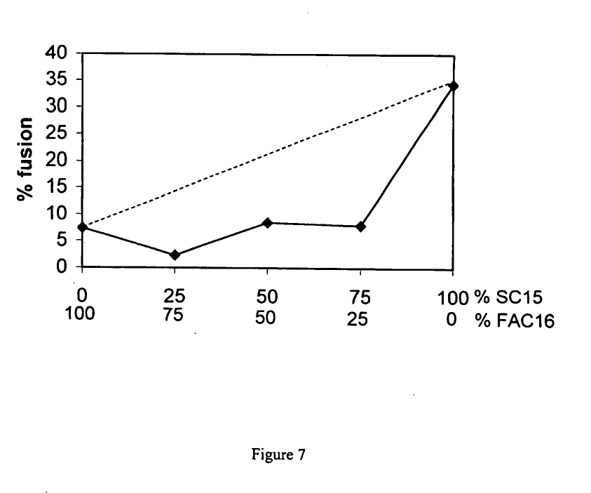

FIG. 7 represents the percentage of charged LUV fusion induced by SIV tilted peptide at a concentration of 0.966 μM in the presence of a mix of SC15 and FAC16 in different molar proportion at a concentration of 1.29 μM in a Tris NaCl buffer at pH 7.4.; the dashed line represents the expected value.

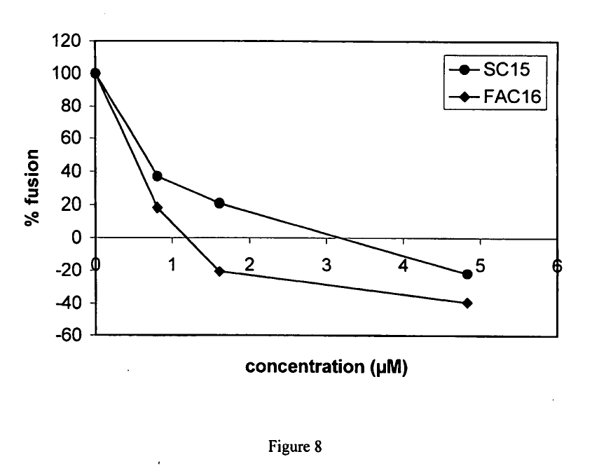

FIG. 8 represents the percentage of charged LUV fusion induced by the Bordetella pertussis tilted peptide at a concentration of 2.42 μM in the presence of SC15 or FAC16 at different concentrations in a Tris NaCl buffer at pH 7.4

### EXAMPLE 1

1. Production of Cyclic Lipopeptides

Bacillus subtilis S 499 is well known from the literature: PEYPOUX et al., in European Journal of Biochemistry, Vol. 202, 101-106, 1991; JACQUES et al., in Applied Biochemistry and Biotechnology Vol. 77-79, 223-233, 1999; AKPA et al., in Applied Biochemistry and Biotechnology, Vol. 91-93, 551-561, 2001; HBID et al., in Applied Biochemistry and Biotechnology, Vol. 57-58, 571-579, 1996, and RAZAFINDRALAMBO et al., in Journal of Chromatography, Vol. 639, 81-85, 1993. This strain was isolated by Dr. Lucien DELCAMBE, (Centre National de Production et d'Etude des Substances d'Origine Microbienne in Liège) from Ituri, Congo.

The strain was grown on a solid rich medium (glucose 2%, peptone 1% and yeast extract 1%) at 30° C. during 48 h. A colony was then used to inoculate 100 ml of optimised medium and grown at 130 rpm (Incubator Shaker Model 625, New Brunswick, N.J., USA), at 30° C. for 8 hours. 250 μl of this pre-culture were then transferred into a 1 l-flask containing 500 ml of optimised medium. This second pre-culture was transferred into a 20 l-fermenter containing 12 litres of optimised medium after 16 h of incubation at 30° C. at 130 rpm.

The composition of the optimised medium (Jacques et al., Appl. Biochem. Biotech., Vol. 77-79, 223-233, 1999) is presented in Table 3.

TABLE 3
Composition of optimised medium

| | | |
|---|---|---|
| Casein peptone N1 (Organo-Technie) | 30 | g/l |
| Saccharose (Raffineries tirlemontoises) | 20 | g/l |
| Yeast extract (Organo-Technie) | 7 | g/l |
| KH₂PO₄ (Merck) | 1.9 | g/l |
| CuSO₄ (Merck) | 0.001 | mg/l |
| FeCl₃.6H₂O (Merck) | 0.005 | mg/l |
| NaMoO₄ (Merck) | 0.004 | mg/l |
| KI (Merck) | 0.002 | mg/l |
| MnSO₄.H₂O (Merck) | 3.6 | mg/l |
| MgSO₄ (Merck) | 0.45 | mg/l |
| ZNSO₄.7H₂O (Merck) | 0.014 | mg/l |
| H₃BO₃ (Merck) | 0.01 | mg/l |
| Citric acid (Merck) | 10 | mg/l |

The fermenter used (BIOLAFITTE) is controlled by an electronic control unit for monitoring and automatically correcting the temperature and the pH. It was equipped with three Rushton turbines (TD4) with a diameter of 10 cm. Their positions from the bottom of the recipient are: 10, 20 and 30 cm.

The fermenter containing the optimised culture medium was sterilised in situ during 30 min at 121° C. The initial fermentation conditions were: temperature: 30° C., pH: 7, stirring: 200 rpm. These parameters were kept constant automatically throughout the fermentation period and, in particular, the pH was kept at 7 by the addition of H₃PO₄ 3N or NaOH 3N.

The aeration rate was initially fixed at 6 l/min. After 7 hours, foam appeared and was continuously collected from the fermenter up to 23 h of culture. During this period, air supply was split between air inlet by the top of the fermenter and air inlet by the base of the fermenter. The collecting foam represented a volume of 1.5 l.

After 23 h of culture, aeration by the base of the fermenter was completely closed, in such a way that the culture is under microaerobic conditions. The fermentation period was 72 h.

At the end of the fermentation, biomass was determined by optical density at 600 nm, dry weight and cell counting. Qualitative and quantitative analysis of lipopeptides in the foam and in the culture medium were performed by HPLC with on-line UV/ELSD (Evaporative Light Scattering Detector) detection.

The HPLC system used is a Waters 2690 Alliance System, with thermostatised auto sampler and column oven, a Waters 996 PDA UV detector and an Alltech ELSD detector. The column is a Waters C:18 Spherisorb, S5 ODS2, i.d. 4.6 mm and length 25 cm. The process conditions are as follows: flow: 1 ml/min, temperature: 30° C., λ_{A}=214 nm; λ_{B}=254 nm and λ_{C}=280 nm (simultaneous detection). The gradient used is given in Table 4, where ACN stands for acetonitrile and TFA for trifluoroacetic acid.

TABLE 4
HPLC gradient for the analysis of lipopeptides

| Time (min.) | 0 | 2 | 26.5 | 28.5 | 30.5 | 55.5 | 60.5 | 65.5 | 90.5 | 92.5 | 100 | 110 | 115 |
|---|---|---|---|---|---|---|---|---|---|---|---|---|---|
| % ACN + | 35 | 35 | 50 | 50 | 60 | 65 | 65 | 85 | 85 | 100 | 100 | 35 | 35 |
| 0.05% TFA | | | | | | | | | | | | | |
| % water + | 65 | 65 | 50 | 50 | 40 | 35 | 35 | 15 | 15 | 0 | 0 | 65 | 65 |
| 0.05% TFA | | | | | | | | | | | | | |

The process is a global determination method, which allows to detect in one run the three families of lipopeptides contained in the culture medium or in the foam (surfactins, iturins and fengycins). This method will be often used to control the percentage of recovery of lipopeptides in all samples. Results are presented in Table 5.

TABLE 5
Biomass and lipopeptide concentrations at the end of the culture
for the culture medium and lipopeptide concentrations in the
collected foam.

| | Culture medium | Foam |
|---|---|---|
| Optical density (600 nm) | 11.8 | Nd |
| Dry weight (g/l) | 5.97 | Nd |
| Cell counting (cfu/ml) | 2.2 10⁸ | Nd |
| Iturin concentration (mg/l) | 344 | 1400 |
| Surfactin concentration (mg/l) | 24 | 2500 |
| Fengycin concentration (mg/l) | 423 | 1500 |

n.d.: not determined
Similar results have been obtained with the strain Bacillus subtilis ATCC 7067.

2. Extraction of Crude Lipopeptide Mixture

The extraction of lipopeptides is performed either on the foam or on the culture medium. The extraction procedure is exactly the same whatever the origin of the sample.

When fermentation is over, the bioreactor is emptied and the culture medium collected and centrifuged at 2740 g during 45 minutes. This allows to remove quite 99% of the Bacillus cells from the culture medium to simplify the further extraction and purification procedures.

The centrifuge supernatant is collected and the centrifuge residues are extracted twice with 50 ml of methanol, to determine the effective concentration of lipopeptides.

Extraction consist of shaking, during 30 minutes, with 50 ml of methanol and further centrifugation. The two methanolic phases are pulled together and evaporated to dryness. The evaporation residue is re-dissolved in a known volume of methanol.

HPLC, with on-line UV/ELSD (Evaporative Light Scattering Detector) detection is performed on the extract and on the centrifuged methanolic phases, according to the method described in Table 4 to control the recovery of lipopeptides.

The supernatant is then passed through an ultrafiltration hollow fibre membrane system. Ultrafiltration plays an important role in the lipopeptides recovery as large volumes of media can be processed rapidly at low cost.

The ultrafiltration process comprises three steps. The first step is called the concentration step. The lipopeptides are present in the solution as micelles, the size of which is greater than 10.000 Da (between 30.000 and 50.000 Da), so that they are easily retained on a hollow membrane fibre which has a cut-off of 10.000 Da. The lipopeptides thus remain in the retentate contained in a tank.

HPLC, with the same apparatus and gradient as presented above, is performed on an aliquot of both the retentate and the permeate in order to control the percentage of lipopeptides in each fraction.

A second step, called diafiltration step is performed. Its principle is to add a known quantity of water in order to get the same value as at the beginning of the concentration step, to the remaining retentate. Filtration according to the same method is performed. The diafiltration process is applied two or three times, to wash out the small molecules, such as salts, small peptides and the like.

The third step is the permeation step. An organic solvent is added (volume/volume) to the retentate to destabilise the micelles in monomers. These monomers are of course smaller (quite about 1.000 Da) and can easily pass through the hollow fibre membrane of 10.000 Da cut-off. The high molecular weigh molecules, such as proteins or carbohydrates are retained in the retentate, while lipopeptides are present in the permeate, which is collected.

Once again HPLC is performed on each fraction, to check the percentage of lipopeptides. The global performance of this system is about 80-85% recuperation. The permeate is then evaporated (using a Büchi like rotavapor) till all the organic solvent has disappeared and then lyophilised (using a freeze dryer at −50° C. under 47.10⁻³ M Pa).

The product obtained at the end of the extraction process is a crude lipopeptide mixture, which is under the form of a powder. This product may find applications in the agro-food industry, oil industry, cosmetics, and pharmacy.

3. Purification

The first purification step allows to obtain fractions of each family of lipopeptides. The second purification step allows to separate each homologous in each family.

the samples are prepared by re-dissolution of the crude lipopeptide mixture (0.2 to 0.7 g) in mQ water (volume from 1.5 up to 3 ml), and placed in an ultrasonic bath, with a power not higher than 120 Watts, during 2-3 minutes.

Purification Technique

The purification process is based on the column chromatography elution system, with octadecylsilicagel (ODS, Varian Bondesil C:18, 40 μm, U.S.A.) as stationary phase. The chromatography system is a flash Chromatography Assembly, provided by Sigma-Aldrich. The work pressure is between 0.5 and 1.5 bars. 30 g of ODS are utilized for purification of 0.2 to 0.7 of crude lipopeptide mixture. These 30 g are poured in the column carefully in order to get a plane surface. Four filter papers Wattman no 1 are placed on top of the phase. The stationary phase is then conditioned with 60 ml of methanol and then with 60 ml of mQ water. After the sample is placed on top of the column, and left to penetrate the gel for about 1-2 minutes. Lipopeptides are than eluted utilizing a gradient of acetonitrile (ACN): water:triflouroacetic acid (TFA). Acetonitrile is HPLC grade provided by Sharlaü, water is mQ Millipore and TFA is pure for synthesis, provided by SDS. The gradient is described below, in Table 6. Fractions of about 10 ml are collected.

TABLE 6
Gradient used in Flash chromatography

| | % ACN | % H₂O | |
|---|---|---|---|
| Composition mix | +0.1% TFA | +0.1% TFA | Volume (ml) |
| 1 | 15 | 85 | 100 |
| 2 | 30 | 70 | 200 |
| 3 | 40 | 60 | 200 |
| 4 | 50 | 50 | 100 |
| 5 | 65 | 35 | 200 |
| 6 | 85 | 15 | 200 |

At the end, 100 ml of pure methanol (HPLC grade, Sharlaü) are passed through the column, in order to remove lipopeptides which could not have been desorbed of the column. An aliquot of each 10 ml fraction is passed on HPLC, to determine in which tubes lipopeptides are present. Tubes containing lipopeptides of a same lipopeptide family (either surfactin, or iturin of fengycin) are than pulled together, evaporated and lyophilised.

The three products obtained are respectively an iturins mixture, a fengycins mixture, and a surfactins mixture, purified up to 85-90%.

The homologous molecules of cyclic surfactins (SC13, SC14 and SC15) and fengycins (FAC16) are respectively isolated from the above-mentioned surfactins mixture and fengycins mixtures by reversed phase chromatography using a Chromspher 5 μm C18 column (1×25 cm, Chrompack, Middelburg, The Netherlands).

The following conditions are used: flow rate at 4 ml/min, ACN:H₂O:TFA 0.05% as mobile phase under isocratic conditions for surfactin (85% ACN+0.05% TFA) and under the following gradient for fengycins:

| | Time (min) | | |
|---|---|---|---|
| | 0 | 30 | 43 |
| Acetonitrile + 0.05% TFA | 60 | 65 | 65 |

The detection is performed at 214 and 280 nm simultaneously.

Fractions are collected by hand in glass tubes. Several injections are carried out before pooling the similar fractions. Evaporation of the solvent is performed with a Büchi like rotavapor. Fractions are then lyophilised and submitted to purity control tests.

At the end of this step, four different compositions containing each one of the following molecules are obtained:

SC13: cyclic surfactin with an iso-branched β-hydroxyled fatty acid chain containing 13 carbon atoms

SC14: cyclic surfactin with a linear β-hydroxyled fatty acid chain containing 14 carbon atoms

SC15: cyclic surfactin with an iso-branched β-hydroxyled fatty acid chain containing 15 carbon atoms

FAC16: cyclic fengycin A with a β-hydroxyled fatty acid chain containing 16 carbon atoms

Each of these homologous is a product with a very high added value.

4. Characterisation

Families and homologous of lipopeptides are then characterised. This characterisation is carried out by four different methods
1) HPLC UV/ELSD characterisation by co-chromatography with standards of each family. The method used has already been described in table 4.
2) Surface tension measurement, using either a Tensimat™ or a Lauda TVT. Lipopeptides are solubilised in an aqueous buffer solution (pH 8.5 for surfactins, pH 7.0 for iturins A and fengycins). Several dilutions (10, 50, 100, 200, 500, 1000 and 10000 times) are also needed to establish the profile characteristic curve of the surface tension versus the dilutions. These curves are then compared with references products.
3) Composition in amino acids. Chemical formulas of the cyclic lipopeptides rings are well known, and the number of amino acids residues are well defined for each family.

An acidic hydrolysis with HCl 6.0 N+0.1% phenol is carried out. 1 mg of raw material are weighted in a Sovirel screw-capped tube. Nitrogen is injected in continuous to saturate the atmosphere in the tube (no air is allowed to enter the enceinte). The tubes are then heated at 110° C. during 24 hours.

After 24 hours, tubes are taken out of the heater and placed in pilled ice. The remaining HCl is evaporated under vacuum. In the cooled tube, an known volume of norleucine is then added at a known concentration. The tubes are vortexed for 10 seconds and centrifuged 5 minutes at 7825 g. An aliquot of the supernatants are then collected and adjusted to pH 2.2 with NaOH 7.5 N, in a 5 ml HPLC vial. When pH is 2.2 is reached, a citrate buffer (also pH 2.2) is added to obtain a final concentration of norleucine round 500 nM/ml.

The sample is then filtered on 0.2 μm (Gelman, 0.2 μm filters) and 20 μl are injected automatically in the amino acids analyser (Stein & Moor, Biochrom 20 Plus, Pharmacia-Amersham) and revealed with ninhydrin. Detection wavelengths are 440 nm and 570 nm. The % of amino acids residues are calculated and compared to the ones of the literature.
4) Maldi-TOF analysis as characterisation technique is performed on the samples, according to the method described by Williams et al., 2000 in J. Mass Spectrom., 37, 259-264.

It is important to note that, although—as described above—the lipopeptides have been obtained by extraction and purification after biosynthesis, each of the lipopeptides homologous which, according to the present invention, may be used as anti-tilted-peptide agent may also be obtained either by chemical synthesis, or by chemical modifications of biosynthetised lipopeptides.

### EXAMPLE 2

1. Preparation of Linear Lipopeptides by Chemical Modification

The chemical modification preparation uses separate solutions of SC13, SC14 or SC15 in the HPLC solvents obtained as described above at the end of the purification step of the homologous molecules of cyclic surfactins by reversed phase chromatography. Each solution is neutralised. The organic phase is evaporated on a Büchi-like rotovapor and milli-Q water is added.

The extraction of cyclic lipopeptides from the aqueous is performed by solid phase extraction, with a 20 ml cartridge containing 5 g octadecylsilicagel as solid phase (Bond elut, varian, CA., USA). The phase is first conditioned with 20-50 ml methanol, 20-50 ml water. After adsorption of the product, the phase is washed with 20 ml milli-Q water and eluted with 20 ml methanol.

The eluate is reduced to 8 ml with a Büchi like rotavapor, and 12 ml of a 0.1 N NaOH solution are added. The reaction is performed in a sealed tube at 37° C. during 14 to 22 hours.

HCl 1M is added to stop the reaction until having a pH of 9. The methanol contained in the solution is evaporated. Linearised lipopeptides are then extracted on solid phase and eluted with methanol as described above.

HPLC, with on-line UV detection is performed on the methanolic solution in order to check the result of the linearisation. The conditions used are: flow rate at 1 ml/min; column: Zorbax C18 35 μm 0.46×15 cm (Agilent, Wilmington, Del., USA) ; detection is performed at 214 nm and 254 nm; the following gradient is used as mobile phase.

| | Time (min) | | |
|---|---|---|---|
| | 0 | 30 | 35 |
| Acetonitrile + 0.05% TFA | 60 | 85 | 85 |
| Water + 0.05% TFA | 40 | 15 | 15 |

Each linearised surfactin is purified by preparative HPLC using a Chromspher 5 μm C18 column (1×25 cm, Chrompack, Middelburg, The Netherlands). The detection is performed at 214 nm and 254 nm, and the other conditions are in the following table.

| | Time (min) | | |
|---|---|---|---|
| | 0 | 1 | 22 |
| Flow rate (ml/min) | 0.5 | 4.5 | 4.5 |
| Acetonitrile + 0.1% TFA | 70 | 70 | 84 |
| Water + 0.1% TFA | 0 | 30 | 16 |

Similar fractions from several injections are pooled, evaporated and lyophilised.

Products Obtained Are

LSC13: linear surfactin, with an iso branched β-hydroxylated fatty acid chain containing 13 carbon atoms and with a linear peptide part.

LSC14: linear surfactin, with an linear β-hydroxylated fatty acid chain containing 14 carbon atoms and with a linear peptide part.

LSC15: linear surfactin, with an iso branched β-hydroxylated fatty acid chain containing 15 carbon atoms and with a linear peptide part.

2. Preparation of Linear Lipopeptides by Chemical Synthesis

55 mg of Wang resin preloaded with 0.9 μmol/mg Fmoc-L-Leucine (100-200 Mesh; Advanced Chemtech, Louisville, Ky., USA) is put in the reaction well of a 348 MPS synthesiser (Advanced Chemtech, Louisville, Ky., USA). The resin is rinsed 3 time with 1 ml methanol (MeOH) and one time with 1 ml N-Methyl-Pyrollidone (NMP).

For each amino-acid addition, the resin and the growing peptide are treated according to the following protocol:
A. Add 1 ml NMP. Shake 1 minute and remove the liquid phase. Do it three times.
B. Add 1 ml piperidine 25% in NMP. Shake 1 to 10 minute and remove the liquid phase. Do it two times.
C. Add 1 ml NMP. Shake 1 minute and remove the liquid phase. Do it two times.
D. Add 1 ml MeOH. Shake 1 minute and remove the liquid phase. Do it three times.
E. Add 1 ml NMP. Shake 1 minute and remove the liquid phase. Do it three times.
F. Add 1.55 ml of a NMP/Dimethylsulfoxyde/Diisopropylethylamine 82/16/2 containing 65 mM of the Fmoc-amino-acid which has to be added (Advanced Chemtech, Louisville, Ky., USA), 65 mM of HOBt (Novabiochem, La Jolla, Calif., USA) and 61 mM TBTU (Alexis corporation, Laufelfingen, Switzerland). Shake 1 hour. Rinse the resin two times with 1 ml NMP. This step is repeated a second time for each amino acid addition.
G. Add 1 ml acetic anhydride 20% in NMP. Shake during 5 minutes and remove the liquid phase. Do it two times.
H. Add 1 ml NMP. Shake 1 minute and remove the liquid phase. Do it three times.
I. Add 1 ml MeOH. Shake 1 minute and remove the liquid phase. Do it three times.

The addition of fatty acid is performed exactly with the same protocol as above as if a eighth amino acid has to be added, except that solution in step F is 130 μM fatty acid, in place of 65 μM Fmoc amino acid.

The resin is then dried, removed from the reaction wells and treated with 1 to 10 ml trifluoroacetic acid (TFA) during 1 hour. Resin is removed by filtration and the solution is dried under nitrogen flow.

The linear lipopeptide is resuspended with milliQ water, shaked, and filtrated. The crude lipopeptide is dissolved in 1-10 ml methanol and HPLC is performed in order to check the composition of the product. The conditions used are: flow rate at 1 ml/min; column: Zorbax C18 35 μm 0.46×15 cm (Agilent, . . . ); detection performed at 214 nm and 254 nm; and the following gradient as mobile phase.

Time (min)

| | 0 | 30 |
|---|---|---|
| Acetonitrile + 0.05% | 20 | 100 |
| TFA | | |
| Water + 0.05% TFA | 80 | 0 |

For linear lipopeptide with a very long acid chain (for example surfactin with 18 carbon atoms), other conditions are used: flow rate at 1 ml/min; column: 214TP104 C4 10 μm 0.46×25 cm (Vydack, Hesperia, Calif., USA) detection performed at 214 nm and 254 nm; and the following gradient as mobile phase.

Time (min)

| | 0 | 30 |
|---|---|---|
| Acetonitrile + 0.05% | 0 | 100 |
| TFA | | |
| Water + 0.05% TFA | 100 | 0 |

Purification of linear lipopeptides is performed by HPLC. Conditions are adapted to the length and formula of the chain.

For Instance,

to purify linear surfactin with 10 carbon atoms, the following conditions are used. Column: Chromspher 5 μm C18 column (1×25 cm, Chrompack, Middelburg, The Netherlands). Flow rate: 4.5 ml/min. The detection is performed at 214 nm and 254 nm.

Time (min)

| | 0 | 30 |
|---|---|---|
| Acetonitrile + 0.1% TFA | 55 | 70 |
| Water + 0.1% TFA | 45 | 30 |

to purify linear surfactin with 18 carbon atoms, the following conditions are used. Column: 214TP1022 C4 10 μm 2.2×25 cm (Vydack, Hesperia, Calif., USA). Flow rate: 23 ml/min. The detection is performed at 214 nm and 254 nm.

Time (min)

| | 0 | 20 |
|---|---|---|
| Acetonitrile + 0.05% TFA | 1 | 25 |
| MeOH/H₂O/TFA | 99 | 75 |
| 82/18/0.05 | | |

Similar fractions are pooled, evaporated and lyophilised.

Surfactin Products Obtained are

LSSC8 to LSSC22: linear synthesised surfactin with a non-branched, non-hydroxyled fatty acid chain containing 8 to 22 carbon atoms and with a linear peptide part.

The characterization of the linear lipopeptides are performed as described for the cyclic lipopeptides. Moreover, infrared spectroscopy is performed using a Brucker IFS 48 spectrometer (Karlsruhe, Germany). 1 mg of product is crushed with 100 mg anhydrous kalium bromide. The mixture is dried and pressed to form a pellet. The spectrum is taken and compared with reference products.

### EXAMPLE 3

Effects of Lipopeptide Preparations on Membrane Fusion Induced by a Tilted Peptide

A number of different systems have been developed to assay peptide membrane fusion activity. Large unilamellar vesicles (LUV) are appropriate model for biological membrane fusion. Their curvature and their stability mimic suitably the cell membrane. Several different techniques for detecting and quantifying vesicle fusion are in common use. These include fluorescence assays that monitor fusion-induced lipid mixing between the two lipid bilayers of the membranes by observing the increase of fluorescence of a probe included in a part of vesicles in mixture. The dilution of the probe by fusion of the two bilayers from the two categories of vesicles causes a decrease of the self-quenching phenomenon leading to an increase of the fluorescence intensity.

Fusion requirements for simple membrane model systems such as LUV are far from those known to be required for biological membranes. However, such studies undoubtedly contribute to a molecular description of different steps of the fusion process.

1. Large Unilamellar Vesicles (LUV) Preparation

Two sets of LUV are prepared: LUV with fluorescent probe (labelled LUV) and free probe LUV (unlabelled LUV). The probe used is the octadecyl rhodamine chloride (R18) (Molecular probes, Eugene, Oreg.).

The first step is the preparation of large multilamellar vesicles. For this, a mixing of different lipids is operated in a round bottomed Büchi flask. Two lipid compositions are tested: a composition with charged lipids and a composition with uncharged lipids. Charged LUV contain phospholipids such as phosphatidylinositol (PI) and phosphatidylserine (PS) that are electrically charged. The charged lipid composition renders the LUV model closer to biological membranes. The lipid composition, their molar ratio, the concentration of the lipid stock solutions prepared in chloroform/methanol (2/1) solvent and the taken volume are presented in tables 7 and 8, for charged LUV and uncharged LUV respectively.

TABLE 7
Lipid composition of charged LUV, their molar ratio, the concentration
of the lipid stock solutions and the taken volume.

| | | | Vol- | Vol- |
|---|---|---|---|---|
| | | | umes | umes |
| | | Stock | for free | for |
| | | concen- | probe | labelled |
| | Molar | tration | LUV | LUV |
| Lipid composition | ratio | (mg/ml) | (μl) | (μl) |
| Egg phosphatidylcholine (PC) | 6 | 10.00 | 511.8 | 321.6 |
| (Sigma, St. Louis, MO, USA) | | | | |
| Egg phosphatidylethanolamine | 6 | 17.86 | 280.4 | 175.9 |
| (PE) (Lipid products, redhill, | | | | |
| Surrey, UK) | | | | |
| Phosphatidylinositol (PI) (Lipid | 0.5 | 10.00 | 46.9 | 29.4 |
| products, Redhill, Surrey, UK) | | | | |
| Phosphatidylserine (PS) | 2 | 10.00 | 165.2 | 105.6 |
| (Avanti) | | | | |
| Sphingomyelin (SM) (Sigma, | 1 | 10.00 | 80.0 | 50.0 |
| St. Louis, MO, USA) | | | | |
| Cholesterol (Chol) (Sigma, St. | 1.5 | 10.00 | 195.3 | 122.7 |
| Louis, MO, USA) | | | | |

TABLE 8
Lipid composition of uncharged LUV, their molar ratio, the
concentration of the lipid stock solutions and the taken volume.

| | | | | Vol- |
|---|---|---|---|---|
| | | | Volume | umes |
| | | Stock | for free | for |
| | | concen- | probe | labelled |
| | Molar | tration | LUV | LUV |
| | ratio | (mg/ml) | (μl) | (μl) |
| Egg phosphatidylcholine (PC) | 1 | 10.00 | 452 | 282 |
| (Sigma, St. Louis, MO, USA) | | | | |
| Egg phosphatidylethanolamine | 1 | 18.52 | 253 | 161 |
| (PE) (Lipid products, Redhill, | | | | |
| Surrey, UK) | | | | |
| Sphingomyelin (SM) (Sigma, St. | 1 | 10.00 | 428 | 267 |
| Louis, MO, USA) | | | | |
| Cholesterol (Chol) (Sigma, St. | 0.76 | 10.00 | 174 | 110 μl |
| Louis, MO, USA) | | | | |

For the labelled LUV, R18 is added at 5.18% and 6.30% of total lipid concentration for charged and uncharged LUV respectively.

The solvent is evaporated by use of a Büchi evaporator. Then, the Büchi flasks are placed in a dessicator during 24 hours. After, the lipid films are wetted by adding 3 and 2 ml of buffer in the free probe LUV flask and the labelled LUV flask respectively. Two compositions of buffer are tested. One is composed by 10 mM Tris (Sigma, St. Louis, Mo., USA), 150 mM NaCl (Merck, Darmstadt, Germany), 0.01% EDTA (Merck, Darmstadt, Germany), 1 mM NaN₃ (Sigma, St. Louis, Mo., USA) with the pH at 8.0 or 7.4, adjusted by HCl or NaCl 1M solution. The other is composed by Tris base 10 mM at pH 8.0. MilliQ water (Water purification Millipore, Molsheim, France) is used to prepare the solutions. The flasks are then dived in a 37° C. bain-marie during one hour and agitated each 15 minutes.

In a second step, large unilamellar vesicles (LUV) are prepared from large multilamellar vesicles. For this, large multilamellar vesicles solutions are transferred from flasks to tubes freezing-proof to undergo 5 cycles of freezing/defrosting. The freezing is performed by plunging the tubes in liquid nitrogen during one minute and the defrosting is performed by plunging the tubes in a 37° C. bain-marie during about 2 minutes. After, each solution is extruded 10 times on a Lipex Biomembranes Extruder (Vancouver, Canada) through one prefilter and two stacked polycarbonate membranes with a pore size of 0.1 μm (Polycarbonate filters Lipex Biomembranes, Vancouver, Canada) previously washed 5 times with the buffer.

The exact lipid concentration in the freshly prepared large unilamellar vesicle solution is determined by the following procedure. A 30 μl and a 60 μl aliquot of each LUV sample is placed in assay tubes. A 30 μl and a 60 μl aliquot of the buffer are used for control. Three repetitions are performed.

The aqueous solution is evaporated in an air drier (Dri-block FisherScientific Techne, Cambridge, UK) warmed at 60° C. After cooling, 400 μl of perchloric acid (Perchloric acid 60%, Merck Eurolab, Leuven) is added in each tube. Four controls of the perchloric acid are carried out (400 μl) and four standard solutions constituted by sodium dihydrogen phosphate monohydrate (Merck, Darmstadt, Germany) at 125 μM (400 μl) are also prepared.

All the tubes are then placed during 45 minutes in a sand bath (LHG) preheated at 200° C. A marble is put on each tube to avoid the solution evaporation. After cooling, two milliliters of ammonium heptamolybdate tetrahydrate (Merck, Darmstadt, Germany) and 100 μl of 1-amino-2-hydroxy-naphthalene-sulfonic acid (AANS) (Merck, Darmstadt, Germany) are added in each tube. The tubes are then warmed during ten minutes in a bain-marie at 100° C. The solution is cooling in an ice bath before measuring the absorbance of each solution at 830 nm on a spectrometer lambda 40 UV/VIS (Perkin-Elmer, Norwalk, Conn. USA) with a slit width of 2.00 nm. Experiments were conducted in a 3 ml cuvette with two frosted sides.

The exact lipid concentration is calculated by the following equation:

$$[ Liquid ] = [ \frac{{A}_{Sample} - {A}_{Control buffer}}{{A}_{Standard} - {A}_{Perchloric acid control}} ] \times \frac{0.05}{x} \times \frac{100}{100 - \% Chol}$$

where A_{Sample}, A_{control buffer}, A_{Standard} and A_{perchloric acid control} are the absorbance of the LUV sample, of the buffer control, of the sodium dihydrogenate phosphate standard, and of the perchloric acid control, respectively, x=0.03 for 30 μl aliquot and x=0.06 for 60 μl aliquot, and % Chol is the percentage of cholesterol in the composition of the LUV.

2. Assay of Vesicles Fusion

Vesicles fusion is determined by measuring the fluorescence intensity change resulting from the fluorescence of the R18 probe. Fluorescence is monitored using fluorimeter LS-50B Perkin-Elmer (Norwalk, Conn. USA). Experiments are conducted in a 2 ml cuvette with right angle illumination. Excitation and emission wavelength are set at 590 nm and 560 nm, respectively, employing a slit width of 10 nm. 300 μl of labelled LUV are mixed with 1200 μl probe free LUV.

25 μl of anti-tilted-peptide agent to be tested, in solution in dimethyl sulfoxyde (DMSO, Sigma, St. Louis, Mo., USA) at different concentrations are added. 25 μl of the tilted peptide solution at a fixed concentration in DMSO are then added. The measurement of fluorescence is operated during 15 minutes. For each measurement, a blanco where the unlabelled LUV are replaced by buffer is carried out. This curve is substracted to the measurement curve. The maximal fluorescence intensity of the resulting curve is taken as data. The percentage of vesicle fusion is calculated by the following equation:

$$\% fusion = ( \frac{{I}_{sample} - {I}_{DMSO}}{{I}_{tilted peptide} - {I}_{DMSO}} ) \times 100$$

where Iₛₐₘₚₗₑ, I_{DMSO}, I_{tilted peptide} are the maximal intensity of fluorescence of the mix of LUV with anti-tilted-peptide agent and tilted peptide, of the mix of LUV with DMSO and of the mix of LUV with tilted peptide and DMSO, respectively.

The 0% vesicle fusion is determined by measuring the fluorescence of the mix of LUV with 50 μl of DMSO. The 100% vesicle fusion is determined by measuring the fluorescence of the mix of LUV with 25 μl of SIV tilted peptide and 25 μl of DMSO.

A control of the absence of fusion (data not shown) in presence of anti-tilted-peptide agent alone is carried out.

3. Results

The ability of tilted peptide to induce intervesicular lipid mixing of LUV is demonstrated by measuring the increase of the fluorescence intensity of the R18 occurring during mixing of fluorescently labelled and unlabelled population of vesicles. The dilution of the probe during the mixing induces the decrease of the self-quenching phenomenon existing when the R18 probe is highly concentrated in the lipid medium.

FIG. 1 presents as example the complete curve of fluorescence intensity variation during the 15 minutes of measurement when the tilted peptide SIV or DMSO is added to the two populations of LUV in mixture. The maximum intensity in the SIV peptide curve is 414.2 (arbitrary unit) and 105.7 in the DMSO curve.

As can be seen from FIG. 1, SIV tilted peptide induces pronounced fusion of labelled and unlabelled LUV populations, whereas the fusion of the two populations is very slight when DMSO alone is added. In presence of lipopeptides alone, no fusion is observed (data not shown). This can explain the negative values of the fusion percentage obtained in the different graphs.

An overview of the different sets of experiments carried out according to the present invention with lipopeptides or, as already known from the state of the art, with lysophosphatidylcholine is presented in Table 9.

TABLE 9
the different sets of experiments carried out with
anti-tilted-peptide agents

| LUV | | | Tilted | Anti-tilted- | |
|---|---|---|---|---|---|
| composition | Buffer | pH | peptide | peptide Agent | FIG. |
| Charged | Tris 10 mM | 8 | SIV | SC15, SC14, | 2 |
| | NaCl 150 mM | | | SC13 | |
| | | | | Fengycins | |
| | | | | mixture | |
| | | | | FAC16 | |
| | | | | Lysophos- | |
| | | | | phatidylcholine | |
| | | | | Sigma, | |
| | | | | St-Louis, USA | |
| | | | | (comparative | |
| | | | | example) | |
| Charged | Tris 10 mM | 8 | SIV | SC13 | 3 |
| | NaCl 150 mM | | | FAC16 | |
| | | | | SC13/FAC16 | |
| | | | | (1:1) | |
| Uncharged | Tris 10 mM | 8 | SIV | SC15 | 4 |
| | NaCl 150 mM | | | Fengycins | |
| | | | | mixture | |
| Uncharged | Tris 10 mM | 8 | SIV | SC15 | 5 |
| | | | | Fengycins | |
| | | | | mixture | |
| Charged | Tris 10 mM | 7.4 | SIV | SC15 | 6 |
| | NaCl 150 mM | | | LSC14 | |
| | | | | FAC 16 | |
| | | | | Iturins A | |
| | | | | mixture | |
| | | | | Crude | |
| | | | | lipopeptides | |
| | | | | mixture | |
| Charged | Tris 10 mM | 7.4 | SIV | SC15 | 7 |
| | NaCl 150 mM | | | FAC16 | |
| | | | | SC15/FAC16 | |
| Charged | Tris 10 mM | 7.4 | Bordetella | SC15 | 8 |
| | NaCl 150 mM | | pertussis | FAC16 | |

SIV tilted peptide has been obtained from SYNT:EM (Nimes, France). This tilted peptide has the amino acid sequence GVFVLGFLGFLA, and induces viral fusion with the host cell. The tilted peptide of Bordetella pertussis has been obtained from EPYTOP (Nimes, France), has the sequence MNTNLYRLVFSHVRGMLV and is part of the signal sequence which causes the secretion of a protein involved in the whooping cough disease.

FIG. 2 represents the percentage of vesicles fusion as a function of anti-tilted peptide agent concentration in the case of charged LUV in a Tris NaCl buffer at pH 8.0. For all antifusogenic agents tested, their addition to the medium results in a concentration dependent inhibition of the LUV fusion. The fusion percentage in presence of lipopeptides falls sharply at concentration below 1 μM.

For fengycins mixture and FAC16, the inhibition is superior to 60% at 3.22.10⁻¹ μM. The complete inhibition is reached at concentration near 1 μM.

For the three surfactins, the inhibition reaches 40-50% at concentrations around 1 μM. For the surfactins category, the complete inhibition is dependent on the chain of the fatty acid. The SC15 and SC14 inhibit nearly completely the fusion at concentration near 2.4 μM whereas SC13 inhibits the fusion at concentration superior to 4.8 μM. At these concentrations, lipopeptides do not induce lysis of the vesicles since no increase of the fluorescence is observed when they are added without tilted peptide in the medium (data not shown).

Lysophosphatidylcholine is known from the literature (Martin, I. and Ruysschaert J-M, Biochimica et Biophysica Acta 1240 (1995) 95-100) to have an antifusogenic effect. It has therefore been tested as comparative example. One can see that at concentrations below 1 μM, lysophosphatidylcholine has no effect at all on the fusion percentage. A slight antifusogenic activity (<50%) exists at concentrations between 1 and 2 μM. It is between 1.5 and 3.5-fold lower than surfactins activity and between 2 and 5-fold lower than fengycins activity.

The mix of lipopeptides from two different families (SC13 and FAC16) exerts also an inhibition on the fusion (FIG. 3). A synergistic effect between the two molecules exists, the value for the mix being inferior to the expected one, which is represented on the Figure by a white rectangle.

The absence of charge in the lipids composing the LUV does not impede the antifusogenic activity of SC15 and fengycins mixture towards the fusion induced by SIV tilted peptide in a Tris NaCl buffer at pH 8.0 (FIG. 4). The fengycins mixture and the SC15 inhibit completely the fusion at concentration near 1 μM and near 3.2 μM, respectively.

The absence of NaCl in the buffer does not impede the antifusogenic activity of SC15 and fengycins mixture when the other conditions are the same (FIG. 5). The activity of SC15 is even slightly improved. Complete inhibition is reached for concentrations near 2 μM.

At the physiological pH (pH 7.4), the behaviour of SC15, FAC16 and iturins A mixture towards the fusion of charged LUV induced by SIV tilted peptide is similar (FIG. 6). At the concentration of 0.8 μM the inhibition is superior to 60%. It reaches 90% in the case of FAC16. The inhibition is complete for concentration near 2.4 μM. The antifusogenic activities of crude lipopeptides mixture and LSC14 are slightly lower. At concentration near 2 μM the inhibition is 40% for the crude lipopeptides mixture and 60% for LSC14.

In these conditions, the mix of SC15 and FAC16 in different molar ratio develops a synergistic effect on the inhibitory activity (FIG. 7). The antifusogenic activity of the mix at all tested percentages is around 3-fold higher than the expected one, which are represented by the dashed line.

As indicated above, tilted peptide may be found in various types of proteins and viruses. Recently, it has been shown by the inventors that the bacterium Bordetella pertussis which is involved in the whooping cough disease comprises a tilted peptide.

FIG. 8 show example of lipopeptides inhibition activity towards the fusion induced by the tilted peptide of Bordetella pertussis. The fusogenic effect of the Bordetella pertussis tilted peptide is inhibited completely by FAC16 at concentration near 1.61 μM and by SC15 at concentration near 4.8 μM.

According to the invention, it is surprisingly shown that lipopeptide preparations may be successfully used as anti-tilted peptide agents against tilted agents of different origins.

1. A method of using lipopeptide preparations as anti-tilted-peptide agents.

2. The method according to claim 1, wherein the lipopeptide preparations comprise lipopeptides which are selected from the group consisting of cyclic and linear lipopeptides, their homologs and derivatives and mixtures thereof.

3. The method according to claim 2, wherein the cyclic lipopeptides are selected from the group consisting of surfactins, iturins and fengycins.

4. The method according to claim 3, wherein surfactins have formula (I) 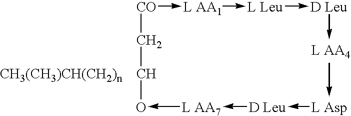
wherein the total number of carbon atoms in the fatty acid chain is comprised between 12 to 17, n being comprised between 6 and 11, AA₁ is Glu or Gln, AA₄ is Val or Ala and AA₇ is Val, IIe or Leu.

5. The method according to claim 3, wherein the surfactins are selected from the group consisting in a surfactin wherein n is comprised between 7 and 9, AA₁ is Glu, AA₄ is Val and AA₇ is Leu.

6. The method according to claim 5 wherein the surfactins are selected from the group consisting of an iso-branched, β-hydroxylated fatty acid chain containing 13 carbon atoms (SC 13), a surfactin with a linear β-hydroxylated fatty acid chain containing 14 carbon atoms (SC14), and a surfactin with an iso-branched β-hydroxylated fatty acid chain containing 15 carbon atoms (SC15).

7. The method according to claim 3, wherein the iturins have formula (II) 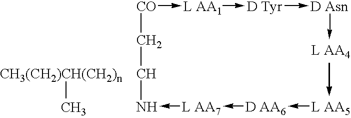
wherein the total number of carbon atoms in the fatty acid chain is comprised between from 13 to 17, n being comprised between 6 and 10, AA₁ is Asn or Asp, AA₄ is Gln, Pro or Ser, and AA₅ is Pro, Glu, or Gln, AA₆ is Asn or Ser, and AA₇ is Ser, Asn or Thr.

8. The method according to claim 3, wherein the iturins are selected from the group consisting in an iturin wherein n is comprised between 7 and 10, AA₁ is Asn, AA₄ is Gln, AA₅ is Pro, AA₆ is Asn and AA₇ is Ser.

9. The method according to claim 8 wherein the iturins are selected from the group consisting of a linear β-amino fatty acid chain containing 14 carbon atoms (IC14), an iturin with an iso-branched β-amino fatty acid chain containing 15 carbon atoms (IC15), an iturin with an iso-branched or linear β-amino fatty acid chain containing 16 carbon atoms (IC16), an iturin with an anteiso-branched β-amino fatty acid chain containing 17 carbon atoms (IC17).

10. The method according to claim 3, wherein fengycins have formula (III) 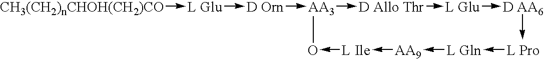
wherein the total number of carbon atoms in the fatty acid chain is comprised between from 12 to 18, n being comprised between 8 and 14, AA₃ is D Tyr or L Tyr, AA₆ is Val or Ala, and AA₉ is L Tyr or D Tyr.

11. The method according to claim 10, wherein fengycin is fengycin A with a β-hydroxylated fatty acid chain containing 16 carbon atoms (FAC 16), wherein AA₃ is D Tyr, AA₆ is Ala and AA₉ is L Tyr.

12. The method according to claim 2, wherein the linear lipopeptides are selected from the group consisting of surfactins, iturins and fengycins.

13. The method according to claim 12, wherein each of the linear lipopeptides is obtainable by chemical modification of the corresponding cyclic lipopeptide.

14. The method according to claim 13, wherein the linear lipopeptides (LSC12 to LSC17 have formula (IV) 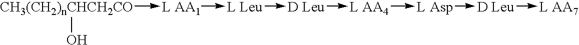
wherein the total number of carbon atoms in the fatty acid chain is comprised between 12 and 17, n being comprised between 8 and 13.

15. The method according to claim 12, wherein each of the linear lipopeptides is obtainable by chemical synthesis.

16. The method according to claim 15, wherein each of the linear lipopeptides (LSSC4 to LSSC24) has the formula (V) 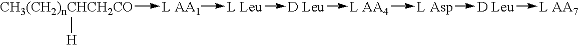
wherein the total number of carbon atoms in the fatty acid chain is 4 to 24; n being comprised between 0 and 20.

17. The method according to claim 14, wherein AA₁ is Glu or Gln, AA₄ is Val or Ala and AA₇ is Val, IIe or Leu.

18. The method according to claim 17, wherein AA₁ is Glu, AA₄ is Val and AA₇ is Leu.

19. The method according to claim 1, wherein the lipopeptide preparations comprise at least two lipopeptides.

20. The method according to claim 19, wherein the lipopeptides belong to different lipopeptide families.

21. The method according to claim 20, wherein one of the lipopeptides is selected from the group consisting of SC13 and SC15 and the other lipopeptide is FAC16.

22. The method according to claim 1, wherein the lipopeptides have been obtained by a method chosen from biosynthesis by a micro-organism, chemical synthesis and chemical modifications of biosynthesized lipopeptides.

23. The method according to claim 22, wherein the micro-organism is selected from the group consisting of Pseudomonas spp, Bacillus spp., Arthrobacter spp, Streptomyces spp., Serratia sp., Gluconbacter spp., and Agrobacterium spp.

24. The method according to claim 23, wherein the species are selected from the group consisting of Bacillus subtillis, Bacillus licheniformis, and Bacillus globigii, Streptomyces aurantiacus, Arthrobacter MIS 38, Serratia marcescens, Gluconobacter cerinu, and Agrobacterium tumefaciens.

25. The method according to claim 24, wherein the Bacillus subtilis is a strain selected from the group consisting of ATCC 7067 and S499.

26. A method of producing a lipopeptide preparation according to claim 22, which comprises an aerobic step followed by a microaerobic step.

27. The method of claim 26, which produces a foam containing a concentrated mixture of different lipopeptide families.
